(19) United States
(12) Patent Application Publication
(10) Pub. No.: US 2026/0124438 A1
(43) Pub. Date: May 7, 2026

(54) NEEDLE PATCH FOR INJECTING DRUG USING LASER

(71) Applicants: Suk Bae SEO, Seoul (KR); Ja Young KIM, Seoul (KR); Ho Joon SEO, Seoul (KR)

(72) Inventors: Suk Bae SEO, Seoul (KR); Ja Young KIM, Seoul (KR); Ho Joon SEO, Seoul (KR)

(21) Appl. No.: 19/426,718

(22) Filed: Dec. 19, 2025

Related U.S. Application Data
(63) Continuation of application No. PCT/KR2024/008530, filed on Jun. 20, 2024.

(30) Foreign Application Priority Data
Jun. 21, 2023 (KR) 10-2023-0079531
Jun. 19, 2024 (KR) 10-2024-0079642

(51) Int. Cl. A61M 37/00; A61L 29/18

(52) U.S. Cl. CPC A61M 37/0092; A61L 29/18; A61M 2037/0007; A61M 2037/0023; A61M 2209/088

(57) ABSTRACT

Provided is a needle patch for injecting a drug using a laser. The needle patch includes an insertion part including a needle inserted into skin, and that is composed of a biodegradable drug dissolved by moisture of the skin and injected into the skin, and a reaction part that is coupled to the insertion part, and generates a shock wave for additionally inserting the needle inserted into the skin by irradiation of a laser beam.

### CROSS-REFERENCE TO RELATED APPLICATIONS

The present application is a continuation of International Patent Application No. PCT/KR2024/008530, filed on Jun. 20, 2024, which is based upon and claims the benefit of priority to Korean Patent Application Nos. 10-2023-0079531 filed on Jun. 21, 2023 and 10-2024-0079642 filed on Jun. 19, 2024. The disclosures of the above-listed applications are hereby incorporated by reference herein in their entirety.

### TECHNICAL FIELD

The present disclosure relates to a needle patch for injecting a drug using a laser.

### BACKGROUND

Generally, a needle patch is mainly used for transdermal delivery of a drug.

A transdermal delivery technology of a drug through a needle patch applies the drug while causing very small damage to the skin, so that fear and pain associated with injection needles may be alleviated, and accordingly, it has high applicability in cosmetic and medical industries.

Generally, a needle patch may include a base and a needle that is supported by the base and inserted into the skin to deliver a drug. Here, a plurality of needles may be provided. Furthermore, the needles may be classified into a hollow microneedle, a coated microneedle, and a dissolving microneedle, depending on a method of delivering a drug to the skin.

A hollow microneedle has a separate drug reservoir in an interior thereof, and delivers a drug in the drug reservoir into the skin by a pressure generated when it is inserted into the skin.

A coated microneedle is one whose outer surface is coated with a biodegradable drug, and after it is inserted into the skin, the biodegradable drug coated on the outer surface is dissolved by moisture of the skin.

A dissolving microneedle is one whose needle itself is made of a biodegradable drug, and after it is inserted into the skin, it is dissolved by moisture of the skin. However, a dissolving microneedle has a weaker rigidity than a metal needle because the needle itself is composed of a biodegradable drug.

Accordingly, a needle patch to which a dissolving microneedle is applied has a very shallow depth, by which the dissolving microneedle penetrates into the skin, due to a rigidity of the dissolving microneedle itself.

### DETAILED DESCRIPTION OF THE INVENTION

### Technical Problem

To solve the above-described problems, an objective of the present disclosure is to provide a needle patch for injecting a drug using a laser, by which a depth, by which a needle composed of a biodegradable drug is inserted into the skin, may be increased.

Problems to be solved by the present disclosure are not limited to the problems mentioned above, and other problems that are not mentioned will be clearly understood by those skilled in the art from the following description.

### Technical Solution

A needle patch for injecting a drug using a laser includes an insertion part including a needle inserted into skin, and that is composed of a biodegradable drug dissolved by moisture of the skin and injected into the skin, and a reaction part that is coupled to the insertion part, and generates a shock wave for additionally inserting the needle inserted into the skin by irradiation of a laser beam.

Furthermore, the reaction part may be a pigment material that absorbs a specific wavelength of the laser beam.

Furthermore, the needle patch may further include a base, by which the reaction part is supported, the reaction part may be interposed between the base and the needle, and a plurality of needles may be provided, and the plurality of needles may be disposed in the reaction part at an interval.

Furthermore, the reaction part may have a shape entirely covering one surface of the base, on which the plurality of needles are disposed.

Furthermore, a plurality of reaction parts may be provided, and the plurality of reaction parts may be interposed between the base and the needles.

Furthermore, the insertion part may further include a connector that extends from the needle toward the reaction part, and a total width of the connector and the reaction part may be greater than a maximum width of a reaction area of the reaction part and the laser beam.

Furthermore, a thickness of the reaction part may be smaller than a thickness of the connector.

### BRIEF DESCRIPTION OF DRAWINGS

FIG. 1 is a cross-sectional view illustrating a needle patch for injecting a drug using a laser according to an embodiment of the present disclosure.

FIGS. 2 and 3 are cross-sectional views illustrating a process of injecting a drug into skin through a needle patch for injecting a drug using a laser according to an embodiment of the present disclosure.

FIG. 4 is a cross-sectional view illustrating an example of an insertion part of a needle patch for injecting a drug using a laser according to an embodiment of the present disclosure.

FIGS. 5 and 6 are cross-sectional views illustrating various examples of a reaction part of a needle patch for injecting a drug using a laser according to an embodiment of the present disclosure.

### MODE FOR CARRYING OUT THE INVENTION

The above and other aspects, features and advantages of the present disclosure will become apparent from embodiments to be described in detail in conjunction with the accompanying drawings. The present disclosure, however, may be embodied in various different forms, and should not be construed as being limited only to the illustrated embodiments. Rather, these embodiments are provided as examples so that the present disclosure will be thorough and complete, and will fully convey the scope of the present disclosure to those skilled in the art. The present disclosure may be defined by the scope of the claims.

Unless otherwise defined, all terms (including technical and scientific terms) used herein have the same meaning as commonly understood by those skilled in the art to which the present disclosure pertains. The terms, such as those defined in commonly used dictionaries, should not be interpreted in an idealized or overly formal sense unless expressly so defined herein.

Hereinafter, embodiments of the present disclosure will be described in detail with reference to accompanying drawings.

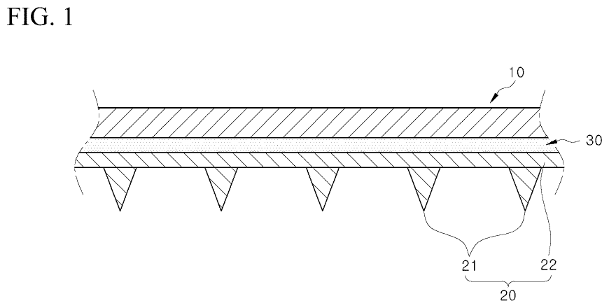

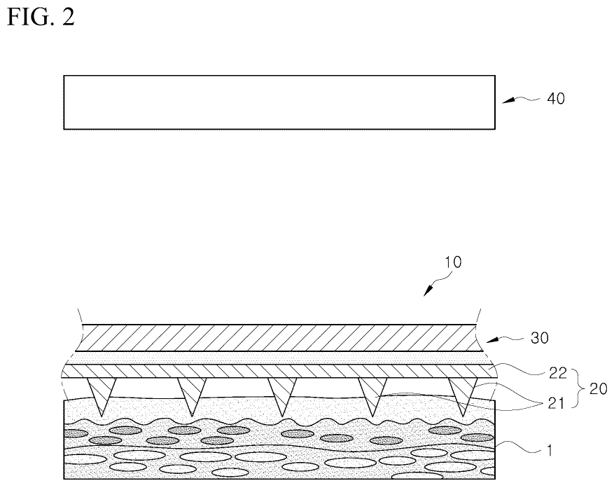

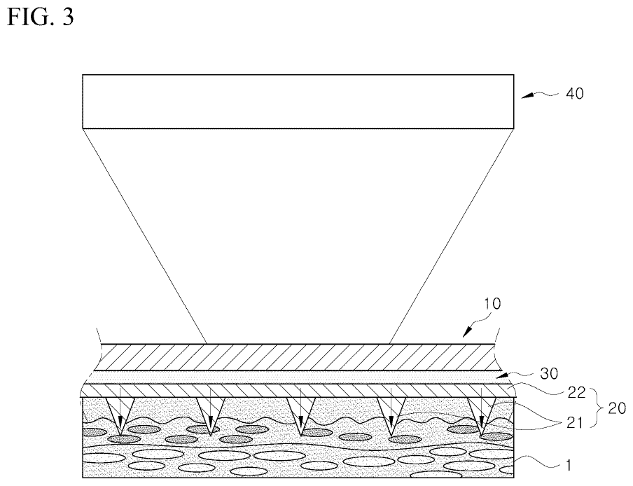

FIG. 1 is a cross-sectional view illustrating a needle patch for injecting a drug using a laser according to an embodiment of the present disclosure, and FIGS. 2 and 3 are cross-sectional views illustrating a process of injecting a drug into skin through the needle patch for injecting a drug using a laser according to an embodiment of the present disclosure.

As illustrated in FIG. 1, a needle patch for injecting a drug using a laser according to an embodiment of the present disclosure includes a base 10, an insertion part 20, and a reaction part 30.

The base 10 may serve to support the reaction part 30. As an example, the base 10 may include an elastic material that is deformed to correspond to a surface of skin 1 and is closely attached to the surface of the skin 1. Here, the elastic material may be at least one of a polyester-based resin such as polyethylene terephthalate and polyethylene naphthalate, a polyethylene resin, a polyvinyl chloride resin, a polycarbonate, a polysulfone, a polyether ether ketone, a polyphenylene sulfide, and a hydrocolloid, or a combination thereof.

The insertion part 20 may serve to inject a drug into the skin 1.

The insertion part 20 may include a needle 21 inserted into skin 1, and a connector 22 that extends from the needle 21 toward the reaction part 30, and may include a biodegradable drug that is dissolved by moisture of the skin 1 and injected into the skin 1. As an example, the biodegradable drug may include hydroxyapatite.

The needle 21 may be inserted into the skin 1.

The needle 21 may be inserted into the skin 1 or, in a state of being inserted into the skin 1, may be dissolved by moisture of the skin 1 and injected into the skin 1 because it is composed of a biodegradable drug.

As an example, a plurality of needles 21 may be provided, and the plurality of needles 21 may be disposed on the connector 22 at intervals. In this case, the connector 22 may serve to connect the plurality of needles 21.

In another example, the plurality of needles 21 may be disposed on the connector 22 in a lattice pattern.

The connector 22 may extend from the needle 21 toward the reaction part 30. The connector 22 may be integrally connected to the needle 21. Furthermore, the connector 22 may be dissolved by moisture on a surface of the skin 1 and permeate into the surface of the skin 1, when contacting the surface of the skin 1.

The reaction part 30 may serve to further insert the needle 21, which is inserted into the skin 1, into the skin 1. Specifically, the reaction part 30 may be coupled to the insertion part 20, and may generate a shock wave by irradiation of a laser beam, through which the needle 21 inserted into the skin 1 is further inserted into the skin 1. As an example, the reaction part 30 may be a pigment material that absorbs a specific wavelength of a laser beam. Here, the pigment material is not particularly limited, and may include at least one of calcium hydroxylapatite, gold nanoparticles, and titanium oxide. A wavelength of the laser beam, with which the pigment material reacts, may be from 500 nm to 3000 nm.

As an example, the reaction part 30 may be accommodated in the base 10.

As another example, the reaction part 30 may be interposed between the base 10 and the needle 31. Here, the reaction part 30 may have a shape that entirely covers one surface of the base 10, on which a plurality of needles 21 are disposed.

For reference, a laser oscillator 40 may serve to irradiate a laser beam to the reaction part 30. A wavelength of the laser beam emitted from the laser oscillator 40 may be from 500 nm to 3000 nm. As an example, the laser oscillator 40 may emit at least one of a nanosecond laser beam, a picosecond laser beam, and a femtosecond laser beam.

Hereinafter, a process of injecting a drug into skin through a needle patch for injecting a drug using a laser according to an embodiment of the present disclosure will be described.

FIGS. 2 and 3 are cross-sectional views illustrating a process of injecting a drug into skin through a needle patch for injecting a drug using a laser according to an embodiment of the present disclosure.

First, the insertion part 20 is moved toward the skin, and the needle 21 is inserted into the skin 1. (see FIG. 2)

Next, the laser beam oscillated from the laser oscillator 40 is irradiated to the reaction part 30. (see FIG. 3)

Subsequently, the reaction part 30 reacts with the laser beam, and a shock wave is generated in the reaction part 30, and the shock wave applies a shock that further inserts the needle 21 inserted into the skin 1. As a result, as the needle 21 is further inserted into the skin 1, a depth, by which the needle 21 is inserted into the skin 1, may be increased.

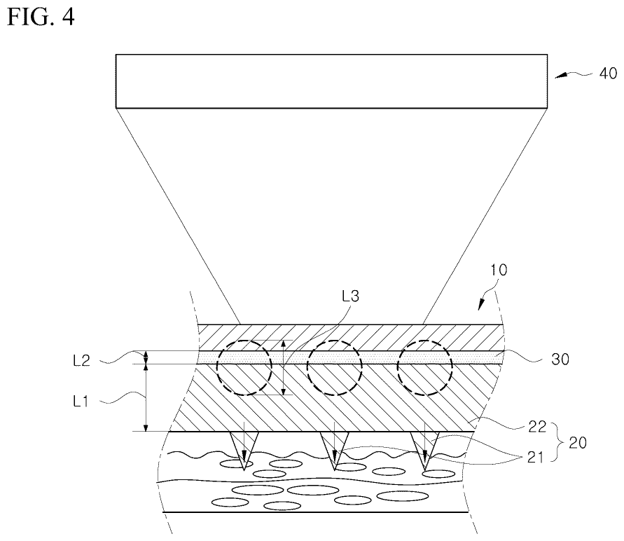

FIG. 4 is a cross-sectional view illustrating an example of an insertion part of a needle patch for injecting a drug using a laser according to an embodiment of the present disclosure.

As illustrated in FIG. 4, total widths L1 and L2 of the connector 22 and the reaction part 30 may be greater than a maximum width L3 of a reaction area of the reaction part 30 and the laser beam. Here, the thickness L2 of the reaction part 30 may be smaller than the thickness L1 of the connector 22.

Accordingly, because a maximum width L3 of a reaction area of the reaction part 30 and the laser beam is not more than the total widths L1 and L2 of the connector 22 and the reaction part 30, it is possible to prevent the reaction part 30 from being deformed in shape or expanded to stick to a surface of the skin 1 when the reaction part 30 reacts with the laser beam while the needle 21 is inserted into the skin 1.

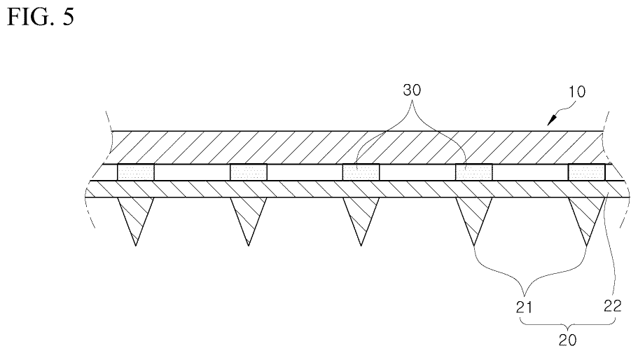

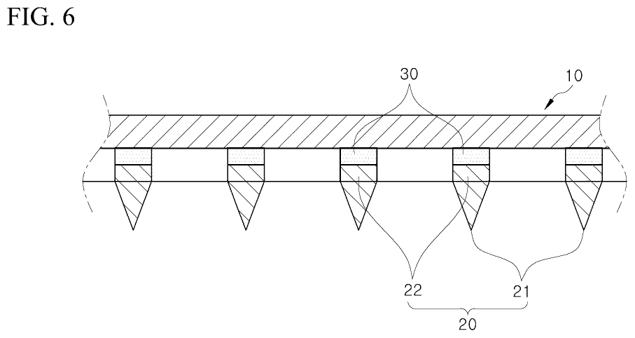

FIGS. 5 and 6 are cross-sectional views illustrating various examples of a reaction part of a needle patch for injecting a drug using a laser according to an embodiment of the present disclosure.

As illustrated in FIG. 5, a plurality of needles 21 may be provided, and a plurality of reaction parts 30 may be provided, and the plurality of reaction parts 30 may be interposed between the base 10 and the plurality of needles 21, respectively.

As illustrated in FIG. 6, a plurality of needles 21 and a plurality of connectors 22 may be provided, and a plurality of reaction parts 30 may also be provided, and the plurality of reaction parts 30 may be interposed between the base 10 and the plurality of connectors 22, respectively.

According to the present disclosure, the needle patch for injecting a drug using a laser according to an embodiment of the present disclosure may increase a depth, by which a needle composed of a biodegradable drug is inserted into the skin.

Effects of the present disclosure are not limited to the effects mentioned above, and other effects not mentioned will be clearly understood by those skilled in the art from the following description.

Although an embodiment of the present disclosure is described with reference to the accompanying drawings, it will be understood by those skilled in the art to which the present disclosure pertains that the present disclosure can be carried out in other detailed forms without changing the scope and spirit or the essential features of the present disclosure. Therefore, the above-described embodiments are exemplary in all aspects, and should be construed not to be restrictive.

1. A needle patch for injecting a drug using a laser, the needle patch comprising:
an insertion part including a needle inserted into skin, and composed of a biodegradable drug dissolved by moisture of the skin and injected into the skin; and
a reaction part coupled to the insertion part, and configured to generate a shock wave for additionally inserting the needle inserted into the skin by irradiation of a laser beam.

2. The needle patch of claim 1, wherein the reaction part is a pigment material absorbing a specific wavelength of the laser beam.

3. The needle patch of claim 1, further comprising:
a base, by which the reaction part is supported,
wherein the reaction part is interposed between the base and the needle, and
wherein a plurality of needles are provided, and the plurality of needles are disposed in the reaction part at an interval.

4. The needle patch of claim 3, wherein the reaction part has a shape entirely covering one surface of the base, on which the plurality of needles are disposed.

5. The needle patch of claim 3, wherein a plurality of reaction parts are provided, and
wherein the plurality of reaction parts are interposed between the base and the needles.

6. The needle patch of claim 1, wherein the insertion part further includes a connector extending from the needle toward the reaction part, and
wherein a total width of the connector and the reaction part is greater than a maximum width of a reaction area of the reaction part and the laser beam.

7. The needle patch of claim 6, wherein a thickness of the reaction part is smaller than a thickness of the connector.
